(19) United States
(12) Patent Application Publication
(10) Pub. No.: US 2006/0241645 A1
(43) Pub. Date: Oct. 26, 2006

(54) Positioning device for a bone cutting guide

(76) Inventor: Stephane Lavallee, Saint Martin D'Uriage (FR)

Correspondence Address: PLEVY & HOWARD, P.C., P.O. BOX 226, FORT WASHINGTON, PA, 19034, US

(21) Appl. No.: 10/560,994

(22) PCT Filed: Jun. 18, 2004
(86) PCT No.: PCT/FR04/50226

§ 371 Date: Jun. 26, 2006

(30) Foreign Application Priority Data
Jun. 18, 2003 (FR) 03/07340

(51) Int. Cl. A61B 17/60

(52) U.S. Cl. 606/96

(57) ABSTRACT

The invention relates a device (10) for the computer-assisted positioning of a cutting guide at the level of a head of bone comprising a base fixable to the bone, a supporting element (80), and means (28, 68, 90) for adjusting the position of the supporting element according to at least three degrees of rotational freedom and two degrees of translational freedom, each adjusting means selectively and continuously adjusting the position according to a degree of rotational and/or translational freedom in an independent manner of other degrees of rotational and/or translational freedom. Said supporting element is used for receiving in a determined manner at least one base (108, 110) which is fixable to the bone and receives the cutting guide, said base being separable from the supporting element.

The present invention relates to a device for positioning a bone-cutting guide.

Some surgical operations, for example, hip or knee arthroplasty, require performing at least one bone cutting at the level of the head of a long bone to arrange a prosthesis. The bone cutting is performed by means of a cutting tool, for example, a miller or an oscillating saw. The cutting tool is maintained in position by a cutting guide attached to the bone by means of a cutting guide positioning device. Generally, several bone cuttings must be performed in distinct planes at the level of a same bone head. A same bone cutting guide is generally used to guide the cutting tool along the different cutting planes. As an example, in the case where the cutting tool is an oscillating saw, the cutting guide may be formed of a single-block piece on which are distributed as many slots as there are cuttings to be performed.

The positioning device generally enables arranging two seats with respect to the bone head. Once positioned, the seats are attached to the head and the positioning device is withdrawn. The cutting guide is then assembled on the seats which ensure a proper positioning of the cutting guide.

The assembly and the setting of the position of the different elements forming the positioning device with respect to one another are generally performed manually based on previously-determined elevations and angles.

The present invention aims at obtaining a device for positioning a cutting guide, said cutting guide being capable of receiving a cutting tool to perform a bone cutting, or several bone cuttings, the positioning device assembly being computer-assisted.

The present invention also aims at obtaining a device for positioning a cutting guide enabling simple and accurate positioning of the cutting guide.

To achieve these objects, the invention provides a device for the computer-assisted positioning of a cutting guide intended to guide a cutting tool capable of cutting bone portions at the level of the head of a bone, comprising base intended to be attached to the bone; a support element; and means for setting the position of the support element according to at least three degrees of rotational freedom and two degrees of translational freedom, each setting means being capable of selectively and continuously setting the position according to a degree of rotational and/or translational freedom in decoupled fashion with respect to the other degrees of rotational and/or translational freedom. The support element is intended to receive in determined fashion at least one seat intended to be attached to the bone and to receive the cutting guide, said seat being detachable from the support element. The support element is intended to receive means for determining the position of the support element.

According to an embodiment of the invention, the setting means are capable of setting the position of the support element according to three degrees of rotational freedom and three degrees of translational freedom.

According to an embodiment of the invention, each of the means for setting the position of the support element according to one degree of translational freedom comprises an element having a threaded opening in which is screwed a threaded rod.

According to an embodiment of the invention, means for setting the position of the support element according to one degree of rotational freedom comprises a pivotal connection and means for blocking said degree of rotational freedom.

According to an embodiment of the invention, the base comprises a cylindrical portion, the device further comprising a first cylindrical piece assembled to freely rotate on the cylindrical portion; means for blocking the first cylindrical piece with respect to the cylindrical portion; a second cylindrical piece assembled to freely rotate and shift on the cylindrical portion; and means for setting the axial position of the second cylindrical piece with respect to the first cylindrical piece.

According to an embodiment of the invention, the device comprises a trolley assembled on the base and two sliding rails capable of pivoting with respect to the trolley around a determined rotation axis and capable of sliding with respect to the trolley according to said determined axis.

According to an embodiment of the invention, the device comprises a threaded rod extending from the trolley along said determined axis, and a thumb wheel screwed on the threaded rod and capable of sliding the sliding rails with respect to the trolley.

According to an embodiment of the invention, the device comprises a first frame arranged in a second frame, the second frame comprising means for guiding the first frame in translation along a determined direction.

According to an embodiment of the invention, the support element is pivotally assembled on the second frame around an axis parallel to the determined direction.

According to an embodiment of the invention, the support element comprises several openings adapted to the assembly of the seat according to a determined configuration from among several determined configurations.

According to an embodiment of the invention, the seat and the cutting guide are solid.

The foregoing objects, features, and advantages, as well as others of the present invention will be discussed in detail in the following non-limiting description of specific embodiments in connection with the accompanying drawings, among which:

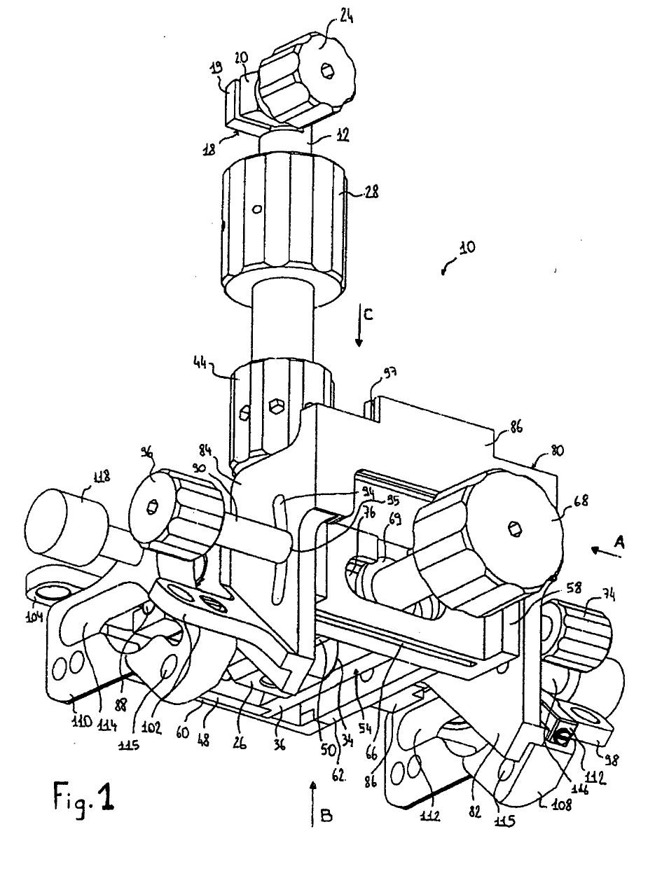

FIG. 1 shows a perspective view of a first example of the forming of the cutting guide positioning device according to the present invention;

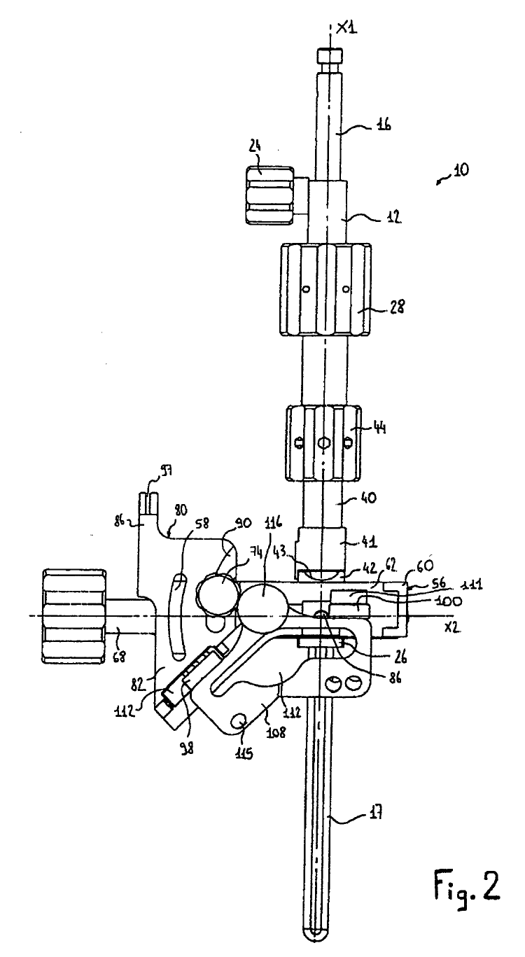

FIG. 2 shows a cross-section view of the positioning device of FIG. 1 along direction A;

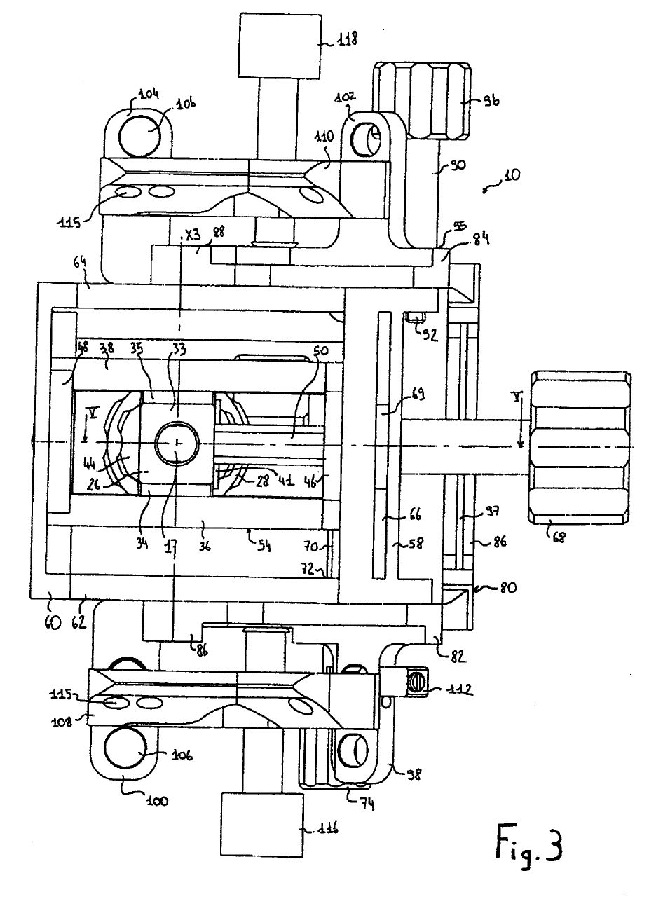

FIG. 3 shows a view of the positioning device of FIG. 1 along direction B;

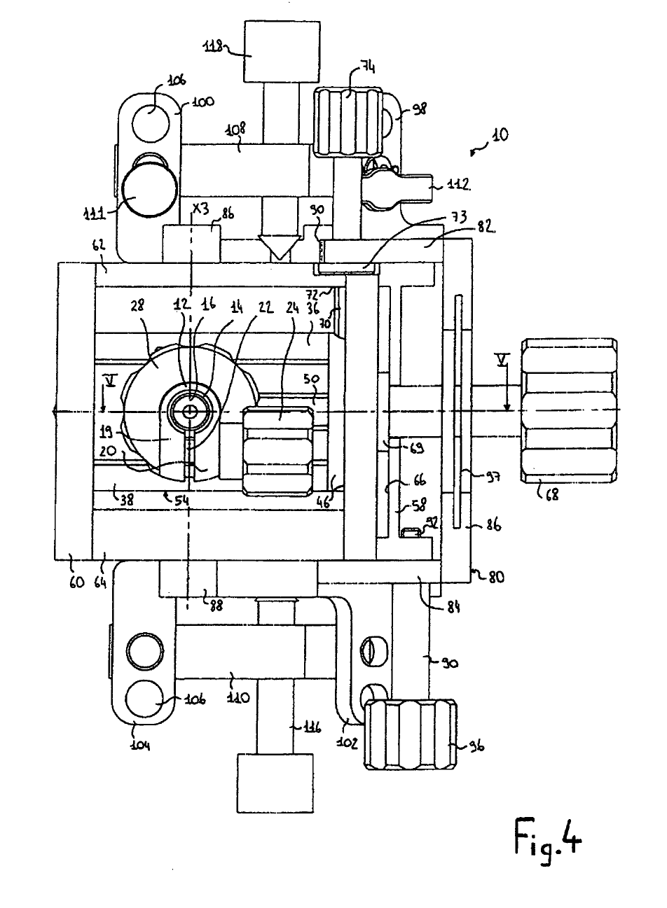

FIG. 4 shows a view of the positioning device of FIG. 1 along direction C; and

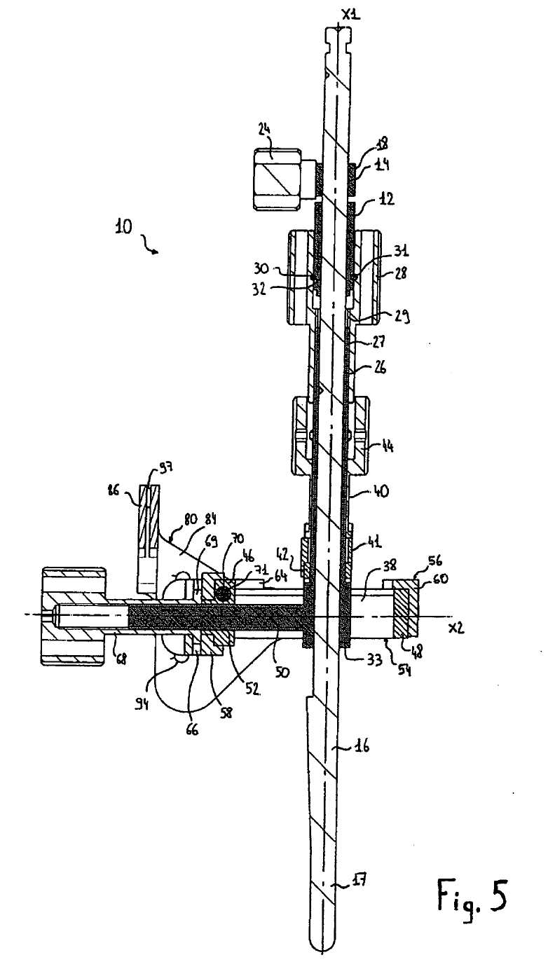

FIG. 5 shows a cross-section view of the device of FIG. 1 substantially along line V-V.

The cutting guide positioning device is intended to be arranged at the level of the head of a long bone, for example, the femur, the tibia, the humerus, etc. The present invention comprises the provision of a positioning device enabling setting of the position of at least one seat intended to receive the cutting guide according to six degrees of freedom, independently from one another. For each translational degree of freedom, the setting is performed by cooperation of two complementary threaded cylindrical pieces having their axes parallel to the shifting direction. Such an arrangement enables fine continuous setting of the relative position between the two pieces according to the translation direction. For each rotational degree of freedom, the setting is obtained by the pivoting of a piece around a rotation axis parallel to the associated translation direction. The pivoting around the rotation axis is prevented by blocking means before and after a setting operation. Such an arrangement also enables fine continuous setting of the pivoting of a piece around the rotation axis.

As shown in FIGS. 1 to 5, device 10 comprises a first cylindrical piece 12 crossed by a cylindrical opening 14 of axis X1. An attachment rod 16 of axis X1 (shown in FIGS. 2 to 5 only) is arranged in opening 14. Rod 16 extends in an anchoring portion 17 intended to be driven into a long bone (not shown) through a head of the bone, substantially along the bone axis. Anchoring portion 17 may comprise anchoring means (not shown) improving the attachment of rod 16 in the bone.

First cylindrical piece 12 is assembled on rod 16 to freely rotate around axis X1. First cylindrical piece 12 can be assembled to freely shift along axis X1 on rod 16. Means for having the first cylindrical piece 12 move along with rod 16 in translation along axis X1 may also be provided. A clamp 18 is arranged at the level of an end of first cylindrical piece 12. Clamp 18 comprises two flanges 19, 20 that may be moved away from or towards each other by a threaded screw 22 actuated by a thumb wheel 24.

Device 10 comprises a second cylindrical piece 26 assembled to freely rotate and shift according to axis X1 on rod 16 substantially in continuation of first cylindrical piece 12. First cylindrical piece 12 comprises fingers (not shown) which extend along axis X1 and cooperate with one or several grooves (not shown) provided at the level of second cylindrical piece 26. Due to the cooperation of the fingers and of the grooves, second cylindrical piece 26 rotates along around axis X1 and freely shifts along axis X1 with respect to first cylindrical piece 12. A threading 27 is formed on a portion of the outer surface of second cylindrical piece 26. A thumb wheel 28, comprising a threaded opening 29 of axis X1, is arranged between first and second cylindrical pieces 12, 26 and is screwed on second cylindrical piece 26. Thumb wheel 28 is held to move along with the first cylindrical piece in translation along axis X1 via a ring 30 arranged in grooves 31, 32 respectively formed on thumb wheel 28 and on first cylindrical piece 12. Ring 30 allow rotation of thumb wheel 28 with respect to first cylindrical piece 12.

When flanges 19, 20 are sufficiently distant from each other, first cylindrical piece 12 can pivot with respect to rotation axis X1 around rod 16 by rotating second mechanical piece 26. The bringing closer of flanges 19, 20 causes the decrease of opening 14, thus ensuring the blocking of first cylindrical piece 12 on rod 16. The rotation of first cylindrical piece 12 and of second mechanical piece 26 with respect to rod 16 around axis X1 is thus prevented.

The rotation of thumb wheel 28 around axis X1 causes the translation of second cylindrical piece 26 along axis X1 with respect to thumb wheel 28, that is, with respect to first cylindrical piece 12 and with rod 16 on which first cylindrical piece 12 is attached by flange 18.

Second cylindrical piece 26 forms, at the end opposite to first cylindrical piece 12, a trolley 33 which comprises two lateral walls 34, 35, each corresponding to a cylindrical portion. Trolley 33 is assembled, at the level of lateral walls 34, 35, in two sliding rails 36, 38, each having an internal wall corresponding to a cylindrical portion so that sliding rails 36, 38 can pivot around trolley 33 around and axis X2, substantially perpendicular to axis X1, and can slide on trolley 33 along axis X2.

Device 10 comprises blocking means 40 screwed on second cylindrical piece 26. A stop 41 is assembled to freely rotate on second cylindrical piece 26 at one end of blocking means 40. A pad 42, having the shape of an arc of a circle, is arranged between stop 41 and trolley 33. Pad 42 is maintained fixed with respect to sliding rails 36, 38 when the latter pivot with respect to trolley 33. Pad 42 thus comprises a groove 43 for the passing of second cylindrical piece 26. Blocking means 40 comprises a thumb wheel 44 to ease its grip. A rotation of thumb wheel 44 causes the shifting of blocking means 40 along axis X1 with respect to second cylindrical piece 26. By coming closer to trolley 33, blocking means 40 pushes stop 41 which presses pad 42 against trolley 33, thus preventing the pivoting of sliding rails 36, 38 with respect to trolley 33.

Sliding rails 36, 38 are maintained at their ends by two planar parallel flanges 46, 48. A threaded rod 50 projects from trolley 33 and extends along axis X2. Threaded rod 50 crosses an opening 52 formed in flange 46. The assembly formed by sliding rails 36, 38 and flanges 46, 48, which will be designated hereafter as the inner frame 54, is arranged in an outer frame 56 formed of a front surface 58, of a rear surface 60, of a first lateral surface 62, and of a second lateral surface 64. Front surface 58 and rear surface 60 are capable of guiding inner frame 54 in translation along a direction X3 perpendicular to directions X1 and X2. Front surface 58 comprises a slot 66 which extends along a plane parallel to directions X1 and X3. A thumb wheel 68 is screwed on threaded rod 50. Thumb wheel 68 extends in a collar 69 arranged in slot 66. When thumb wheel 68 is actuated, it causes the translation of outer frame 56 and of inner frame 54 with respect to threaded rod 50, and thus with respect to second cylindrical piece 26, along axis X2, since thumb wheel 68 is fixed in translation with respect to outer frame 56, due to collar 69.

A threaded rod 70 cooperates with a threaded opening 71 crossing flange 46 along direction X3. First lateral surface 62 comprises a bearing 72 supporting threaded rod 70. A collar 73 maintains threaded rod 70 fixed in translation along direction X3 with-respect to first lateral surface 62. Threaded rod 70 comprises a thumb wheel 74 at the end opposite to flange 46. By actuating thumb wheel 74, threaded rod 70 cooperates with threaded opening 71 of flange 46 and causes the translation along direction X3 of outer frame 56 with respect to inner frame 54. Front surface 58 comprises a groove 76 which extends along direction X3. Groove 76 receives thumb wheel 68 and enables displacement of thumb wheel 68 along direction X3 with respect to outer frame 56.

A support element 80 extends outside of outer frame 56. Support element 80 comprises a substantially planar first support 82 on the side of first lateral surface 62, a second substantially planar support 84 on the side of second lateral surface 64, and a substantially planar connection element 86 connecting the two supports 82, 84 by going round outer frame 56 on the side of front surface 58. First and second supports 82, 84 are substantially symmetrical. Support element 80 is pivotally assembled on outer frame 56 around the rotation axis, confounded hereafter with direction X3, via two bearings 86, 88. First support 82 comprises a groove 90 having the shape of an arc of a circle centered on rotation axis X3 and enabling passing of threaded thumb wheel 70 on pivoting of support element 80 with respect to outer frame 56.

A rod 90 comprising a threaded end 92 is screwed on second lateral surface 64 of outer frame 56 along axis X3. Second support 84 comprises a slot 94 enabling passing of rod 90 on pivoting of support element 80 around axis X3. Rod 90 comprises a shouldering 95 arranged substantially opposite to second support 84. Rod 90 comprises a thumb wheel 96 at one end to ease its grip. By actuating thumb wheel 96, rod 90 can be shifted along axis X3 with respect to second lateral surface 64 outer frame 56 so that shouldering 95 abuts against second support 84 and blocks second support 84 with respect to outer frame 56, thus preventing the pivoting of support element 80 with respect to outer frame 56 around axis X3.

Connection element 86 comprises a slot 97 intended to receive a rigid body (not shown) comprising back-reflective facets. Such a rigid body belongs to a location system (not shown) capable of determining the position of support element 80. The location system for example is of the type comprising a source emitting an infrared radiation and several sensors measuring the infrared radiation reflected by the back-reflective facets. It should be noted that any system for locating support element 80 may be used. As an example, the location system may be based on optical technology (like system POLARIS of NDI Company, Toronto, Canada), based on magnetic technology (like system Fastrack of Polhemus Inc, USA), or based on ultrasound technology (product of Zebris Company, Germany).

Two attachment tabs 98, 100 project perpendicularly to first support 82. Symmetrically, two attachment tabs 102, 104 project perpendicularly to second support 84. Each attachment tab comprises circular and/or oblong openings 106. Positioning device 10 is intended to receive first and second seats 108, 110 of known type. First seat 108 is attached to the two attachment tabs 98, 100 of first support 82. Second seat 110 is attached on the two attachment tabs 102, 104 of second support 84. As an example, an attachment pin 111 and a clamp 112 are shown for the attachment of seat 108 on attachment tabs 98, 100. The different openings 106 provided on the attachment tabs provide several possibilities of assembly of seats 108, 110 on attachment tabs and enable modifying the interval between seats 108, 110.

Each seat 108, 110 comprises in known fashion a main hole 112, 114 to lighten its weight, and several secondary openings 115, intended to receive screws (not shown). A peak 116, 118 with a pointy end is screwed on each seat 108, 110.

An example of implementation of positioning device 10 according to the present invention is the following: positioning device 10 is attached to the bone by insertion of rod 16 along the bone axis. A calculator (not shown) connected to a location system may provide on a display the real position of support element 80 with respect to the bone and theoretical position of support element 80 to be obtained. The calculator may have a mathematical model of positioning device 10 and then provide based on the real and theoretical positions, the settings to be performed according to each degree of freedom of positioning device 10 to pass from the real position to the theoretical position. The operator can check on the display, while performing the settings, the variation of the position of support element 80.

According to a variation of the present invention, electric motors may be assembled on the positioning device to obtain an automatic computer-assisted setting of each degree of freedom.

The cutting guide device according to the present invention has many advantages:

First, the device according to the invention enables positioning a cutting guide with respect to a bone according to six degrees of freedom, the setting being performed independently for each degree of freedom.

Second, for each degree of freedom, the setting can be performed continuously with accuracy.

Third, the positioning device can easily be adapted to different cutting guides, since different types of seats can be attached to the positioning device according to several possible configurations.

Fourth, the positioning of the cutting guide may be computer-assisted, thus easing the setting of the device and improving its accuracy.

Of course, the present invention is likely to have various alterations and modifications which will occur to those skilled in the art. In particular, attachment rod 16 may be replaced with any means for attaching the positioning device to the bone head, for example, a screw mechanism.

1. A device (10) for the computer-assisted positioning of a cutting guide intended to guide a cutting tool capable of cutting bone portions at the level of the head of a bone, comprising:
a base (16) intended to be attached to the bone;
a support element (80); and
means (28, 68, 90) for setting the position of the support element according to at least three degrees of rotational freedom and two degrees of translational freedom, each setting means being capable of selectively and continuously setting the position according to a degree of rotational and/or translational freedom in decoupled fashion with respect to the other degrees of rotational and/or translational freedom,
in which support element is intended to receive in determined fashion at least one seat (108, 110) intended to be attached to the bone and to receive the cutting guide, said seat being detachable from the support element, and in which the support element is intended to receive means for determining the position of the support element.

2. The device of claim 1, in which the setting means (28, 68, 90) are capable of setting the position of the support element (80) according to three degrees of rotational freedom and three degrees of translational freedom.

3. The device of claim 1, in which each of the means (28, 68, 90) for setting the position of the support element (80) according to one degree of translational freedom comprises an element (28, 46, 68) having a threaded opening (71) in which is screwed a threaded rod (26, 50, 70).

4. The device of claim 1, in which at least one means for setting the position of the support element (80) according to one degree of rotational freedom comprises a pivotal connection (86, 88) and means (18, 40, 42, 90) for blocking said degree of rotational freedom.

5. The device of claim 1, in which the base (16) comprises a cylindrical portion, the device further comprising:
a first cylindrical piece (12) assembled to freely rotate on the cylindrical portion;
means (18) for blocking the first cylindrical piece with respect to the cylindrical portion;
a second cylindrical piece (26) assembled to freely rotate and shift on the cylindrical portion; and
means (28) for setting the axial position of the second cylindrical piece with respect to the first cylindrical piece.

6. The device of claim 1, comprising a trolley (33) assembled on the base (16) and two sliding rails (36, 38) capable of pivoting with respect to the trolley around a determined rotation axis (X2) and capable of sliding with respect to the trolley according to said determined axis.

7. The device of claim 6, comprising a threaded rod (50) extending from the trolley (33) along said determined axis (X2), and a thumb wheel (28) screwed on the threaded rod and capable of sliding the sliding rails (36, 38) with respect to the trolley.

8. The device of claim 1, comprising a first frame (54) arranged in a second frame (56), the second frame (56) comprising means for guiding the first frame in translation along a determined direction.

9. The device of claim 8, in which the support element (80) is pivotally assembled on the second frame (56) around an axis parallel to the determined direction.

10. The device of claim 1, in which the support element (80) comprises several openings (106) adapted to the assembly of the seat (108, 110) according to a determined configuration from among several determined configurations.

11. The device of claim 1, in which the seat (108, 110) and the cutting guide are solid.
